SEQUENCE LISTING (2 sequences)

SEQ ID NO: 1 - protein, 22 aa, Artificial Sequence
  Amino acid sequence of the antigen peptide
CSCPYVSYGRFCIQPDGSIKQC

SEQ ID NO: 2 - protein, 20 aa, Artificial Sequence
  Amino acid sequence of the antigen peptide
SCPYVSYGRFCIQPDGSIKQ

(19) United States
(12) Patent Application Publication
(10) Pub. No.: US 2023/0375546 A1
(43) Pub. Date: Nov. 23, 2023

(54) Elisa Method as an Alternative to Neutralization Potency Assay and Use Thereof

(71) Applicant: Yangzhou University, Yangzhou (CN)

(72) Inventors: Aijian Qin, Yangzhou (CN); Yuhan Zhang, Yangzhou (CN); Qi Wu, Yangzhou (CN); Kun Qian, Yangzhou (CN); Hongxia Shao, Yangzhou (CN); Jianqiang Ye, Yangzhou (CN)

(21) Appl. No.: 17/631,528

(22) PCT Filed: May 31, 2021
(86) PCT No.: PCT/CN2021/097238

§ 371 Date: Mar. 17, 2022

(30) Foreign Application Priority Data
Sep. 8, 2020 (CN) 202010937821.3

(51) Int. Cl. G01N 33/569; C07K 14/005; C07K 16/10

(52) U.S. Cl. CPC G01N 33/56983; C07K 14/005; C07K 16/1002; G01N 2333/165; G01N 2469/20; C12N 2770/20022; C07K 2317/34; C07K 2317/76

(57) ABSTRACT

Disclosed is an antigen peptide with an infectious bronchitis virus (IBV)-specific neutralizing epitope, in which the peptide is a cyclic polypeptide, and the amino acid sequence of the peptide is CSCPYVSYGRFCIQPDGSIKQC. Also disclosed are an IBV specific antibody and a preparation method thereof. Further disclosed is an ELISA method as an alternative to the neutralization potency assay. The disclosure also discloses an ELISA detection kit, and using the established pELISA method to detect IBV antibodies, it is found that the method is positively correlated with anti-IBV neutralizing antibodies, which may be used to evaluate the immune effect of IBV vaccines on IBV infected chickens and to determine the antibody level, thereby benefiting the health management of chicken flocks.

### CROSS REFERENCE TO RELATED APPLICATIONS

This patent application is a U.S. National Phase application under 35 U.S.C. § 371 of International Application No. PCT/CN2021/097238, filed on May 31, 2021 and which claims the benefit and priority of Chinese Patent Application 202010937821.3 filed on Sep. 8, 2020, titled “ELISA Method As An Alternative to Neutralization Potency Assay and Use thereof” the disclosure of which is incorporated by reference herein in its entirety as part of the present application.

### TECHNICAL FIELD

The present disclosure belongs to the technical field of biomedicine, and in particular relates to an ELISA method as an alternative to neutralization potency assay and use thereof.

### BACKGROUND ART

Infectious bronchitis (IB) is an infectious disease caused by infectious bronchitis virus (IBV). IBV infection in chickens causes severe respiratory and kidney diseases, leading to significant economic losses. Although the vaccine is now widely used, the prevalence of IB can still be observed in chickens.

Neutralizing antibodies are corresponding antibodies produced when pathogenic microorganisms invade the body. When pathogenic microorganisms invade cells, they need to rely on the specific molecules expressed by the pathogens to bind to the receptors on the cells in order to infect the cells for further multiplying. Neutralizing antibodies are certain antibodies produced by B lymphocytes that can bind to antigens on the surface of pathogenic microorganisms, thereby preventing the pathogenic microorganisms from adhering to target cell receptors and preventing cell invasion. Neutralizing antibody is a soluble protein secreted by adaptive immune cells. After the body is invaded by virus, the adaptive immune cells secrete neutralizing antibodies into the blood, which bind to the virus particles in the blood to prevent the virus from infecting the cells and destroy the virus particles, thus “neutralizing” the virus.

At present, cell culture methods are generally adopted to detect the titers of neutralizing antibodies in serum to evaluate the potency of vaccines in vaccinated chickens. However, this process is time-consuming, laborious and difficult to achieve at the grass-roots level. It has been reported that immunofluorescence and enzyme-linked immunosorbent assay (ELISA) can replace the virus neutralization test, which make it faster and easier to determine the neutralization titer of a serum antibody. Recent studies have shown that ELISA of rabies virus and bovine viral diarrhea virus glycoprotein could determine the neutralizing antibody titers in the serum of vaccinated humans and cattle respectively. The correlation of antibody titers between indirect ELISA and neutralization test has also been studied in Zika virus and human papilloma virus.

Although ELISA methods for detecting antibodies against whole virus particles or recombinant S1 protein, N protein and non-structural proteins have been developed so far, there is no serological method in place of the neutralization test of infectious bronchitis virus.

### SUMMARY

The objective of the disclosure is described herein as follows. The IBV genome encodes 4 main structural proteins, i.e., S protein, E protein, M protein, and N protein, in addition to fifteen non-structural proteins and some accessory proteins. Among these proteins, S glycoprotein is considered to be the main protective antigen carrying neutralizing epitopes and can induce the production of neutralizing antibodies. However, S1 varies in different strains of IBV. S2 is a highly conserved protein that carries a wide range of epitopes, with related neutralizing epitopes presented. The present disclosure also shows that S2 has a broad-spectrum neutralizing epitope.

The present disclosure synthesizes a peptide with the neutralizing epitope, and establishes an ELISA method for detecting IBV antibodies. The method is found to be correlated with the titer of neutralizing antibodies, which can be used for evaluation and determination of neutralizing antibodies.

The serum antibody level detected by the peptide ELISA of the present disclosure is positively correlated with the neutralizing antibody level.

Another technical problem to be solved by the present disclosure is to provide an ELISA method as an alternative to neutralization potency assay, which can be adopted to evaluation of neutralizing antibody level after vaccination.

Another technical problem to be solved by the present disclosure is to provide the use of an antigen peptide with an IBV-specific neutralizing epitope in the preparation of an IBV detection kit.

Yet another technical problem to be solved by the present disclosure is to provide an ELISA kit.

The technical scheme of the present disclosure is described herein as follows. To solve the above problems, the present disclosure provides an antigen peptide with an IBV-specific neutralizing epitope, in which the peptide is a cyclic peptide and the amino acid sequence of the cyclic peptide is CSCPYVSYGRFCIQPDGSIKQC (SEQ ID NO: 1) or SCPYVSYGRFCIQPDGSIKQ (SEQ ID NO: 2).

In some embodiments, the present disclosure includes an IBV-specific antibody and the IBV-specific antibody binds to the antigen peptide with the IBV-specific neutralizing epitope.

In some embodiments, the present disclosure further includes a method for preparing the IBV-specific antibody, in which the method includes mixing the antigen peptide with the IBV-specific neutralizing epitope with an adjuvant to immunize a mouse, a chicken or other experimental animals, and obtaining an IBV-specific antibody after 2-3 immunizations. In an embodiment of the present disclosure, a volume ratio between the antigen peptide with the IBV-specific neutralizing epitope and the adjuvant is 1:1.2. The antigen peptide is synthesized by Shanghai Synpeptide Co., Ltd. An interval between every two immunizations is preferably 10 days. A dose for each immunization is 0.2 mL/individual.

The present disclosure also provides an ELISA method as an alternative to neutralization potency assay, including the following steps:
1) coating the antigen peptide with the IBV-specific neutralizing epitope on an ELISA plate, and blocking it with rabbit serum after coating;
2) after washing for several times, adding chicken serum for co-incubation;
3) washing again for several times, then incubating with an enzyme-labeled goat anti-chicken IgG antibody, and after washing for several times, using tetramethylbenzidine (TMB) substrate to develop color; and
4) terminating the reaction with 0.1% sodium dodecyl sulfate (SDS), and detecting an absorbance at 650 nm with an enzyme-linked immunosorbent analyzer to determine a neutralizing antibody level against IBV in an individual.

In an embodiment of the present disclosure, 200 μL of the rabbit serum is added per well. The washing solution is a phosphate-buffered saline containing Tween-20 (PBST). 100 μL of the chicken serum is added per well. A co-incubation is conducted at a temperature of 37° C. or room temperature in water bath. The co-incubation is conducted for 60 min.

In some embodiments, the present disclosure further includes the use of the antigen peptide with the IBV-specific neutralizing epitope in the preparation of an IBV detection kit.

In some other embodiments, the present disclosure includes an ELISA kit, including the antigen peptide with the IBV-specific neutralizing epitope.

In an embodiment, the ELISA kit includes the following components: an ELISA plate coated with the antigen peptide with the IBV-specific neutralizing epitope, an enzyme-labeled antibody, a substrate solution, a stop solution, a washing solution, a negative control and a positive control.

In an embodiment, the enzyme-labeled antibody is an enzyme-labeled goat anti-chicken IgG antibody.

In an embodiment, the substrate solution is a TMB substrate color developing solution.

In an embodiment, the negative control is specific-pathogen-free (SPF) chicken serum.

In an embodiment, the positive control is serum of a SPF chicken immunized with IBV.

The embodiments may have the following beneficial effects: using the pELISA method established by the present disclosure, antibodies of all types of IBV currently known can be detected, while conventional ELISA can only detect antibodies induced by classic IBV strains, and it is found that this method is positively correlated with anti-IBV neutralizing antibodies. The present disclosure may be adopted to evaluate the immune effect of IBV vaccine and determine the antibody level of IBV-infected chickens, thereby benefiting the health management of chicken flocks and the like.

### BRIEF DESCRIPTION OF THE DRAWINGS

FIG. 1 shows the absorbance at 650 nm detected by an enzyme-linked immunosorbent analyzer after the cyclic peptide reacts with serum samples containing different viruses;

FIG. 2 is a comparison of the correlation between neutralizing antibodies and ELISA antibodies in serum samples immunized with different virus strains;

FIG. 3 shows the good correlation between neutralizing antibodies and ELISA antibodies after M41 and CK/CH/2010/JT1 infected chickens.

### DETAILED DESCRIPTION OF THE EMBODIMENTS

The present disclosure will be further explained below in conjunction with specific examples. It should be understood that these examples are only used to illustrate the present disclosure and not to limit the scope of the present disclosure. In addition, it should be understood that after reading the teachings of the present disclosure, those skilled in the art can make various changes or modifications, and these equivalent forms also fall within the scope defined by the appended claims of the application.

Unless otherwise specified, the reagents, methods and equipment used in the present disclosure are conventional reagents, methods and equipment in the art.

### Example 1

Peptide synthesis: The cyclic peptide CSCPYVSYGRFCIQPDGSIKQC (SEQ ID NO: 1) was synthesized according to a conventional method, and the purity identified by chromatography was greater than 95%.

The peptide SCPYVSYGRFCIQPDGSIKQ (SEQ ID NO: 2) was synthesized according to a conventional method, and the purity identified by chromatography was greater than 95%.

pELISA method: The synthetic cyclic peptide CSCPYVSYGRFCIQPDGSIKQC (SEQ ID NO: 1) and the peptide SCPYVSYGRFCIQPDGSIKQ (SEQ ID NO: 2) were respectively coated on the ELISA plate at a coating concentration of 0.1 pg/mL in a 0.05 M carbonate buffer (pH=9.6), with 100 μL per well, coated overnight at 4° C., and blocked with 8% rabbit serum at 37° C. for 3 hours. After 3 washes with PBST were conducted, 100 μL of anti-IBV chicken serum (1:200 dilution with PBST) was added for incubation at 37° C. for 1 hour, then 5 times of washing were conducted, and then 100 μL of enzyme-labeled goat anti-chicken IgG antibody (product of Jackson) was added for mixing. A resulting mixture was incubated for 1 hour at 37° C. After 5 washes were conducted, 100μL of TMB substrate was used to develop color at 37° C. for 15min. 100 μL of 1% SDS was added to stop the reaction. The absorbance at 650 nm was detected with an enzyme-linked immunosorbent analyzer.

This method is not compatible with positive serum samples against viruses such as avian influenza virus (AIV), avian leukemia virus (ALV), avian reticuloendothelial virus (REV), goose plague virus (GPV), avian infectious bursal disease virus (IBDV), infectious laryngotracheitis virus (ILTV), Marek's Disease Virus (MDV), and chicken egg drop syndrome virus (EDSV), etc. As shown in FIG. 1, the cyclic peptide CSCPYVSYGRFCIQPDGSIKQC (SEQ ID NO: 1) and different sera containing virus were used to detect the absorbance at 650 nm with an enzyme-linked immunosorbent analyzer. A cutoff value between the negative and positive samples was 0.2. It could be seen from the absorbance value at 650 nm in Table 1 below that the OD value of the cyclic peptide was higher, and the sensitivity and specificity were better. The storage life of the peptide SCPYVSYGRFCIQPDGSIKQ (SEQ ID NO: 2) without C was shorter, and the cyclic peptide CSCPYVSYGRFCIQPDGSIKQC (SEQ ID NO: 1) with C was stable and had a longer storage life.

TABLE 1
Detection results of different forms of peptides

| A. Test results of the | | | B. Test results of the | | |
|---|---|---|---|---|---|
| cyclic peptides; | | | peptide | | |
| SPF | 0.048 | 0.047 | SPF | 0.048 | 0.046 |
| M41 | 1.450 | 1.420 | M41 | 0.932 | 0.956 |
| 4/91 | 1.310 | 1.350 | 4/91 | 0.703 | 0.620 |
| FJ14 | 1.500 | 1.490 | FJ14 | 0.871 | 0.897 |
| QL1403 | 1.390 | 1.420 | QL1403 | 0.860 | 0.865 |
| JT-1 | 1.350 | 1.330 | JT-1 | 0.832 | 0.823 |

Clinical sample analysis: Among 250 clinical immunized chicken serum samples (from a breeding company), 233 serum samples were identified positive by pELISA, 17 serum samples were identified negative by immunofluorescence (IFA), 232 serum samples were identified positive by IFA, and 18 serum samples were identified negative by IFA. Compared with IFA, the accuracy of pELISA for 250 serum samples was 98.8%. Therefore, pELISA had very good reliability. The method of IFA detection referred to the prior art (Qi Wu, Zhixian Lin, Jinsen Wu, Kun Qian, Hongxia Shao, Jianqiang Ye and Aijian Qin. Peptide enzyme-linked immunosorbent assay (pELISA) as a possible alternative to the neutralization test for evaluating the immune response to IBV vaccine. BMC Veterinary Research (2021) 17:51)

TABLE 2
Comparison of pELISA and IFA test results

| | | IFA | | |
|---|---|---|---|---|
| | | positive | negative | number |
| pELISA | positive | 231 | 2 | 233 |
| | negative | 1 | 16 | 17 |
| | number | 232 | 18 | 250 |

TABLE 3
Stability test results of pELISA

| | Difference | | Differences | |
|---|---|---|---|---|
| | within a batch | | between the batches | |
| Replicates | SD | CV(%) | X ± SD | CV(%) |
| 1 | 0.043 ± 0.001 | 2.3% | 0.048 ± 0.001 | 2.7% |
| 2 | 0.046 ± 0.001 | 1.6% | 0.046 ± 0.001 | 1.9% |
| 3 | 0.049 ± 0.001 | 2.3% | 0.051 ± 0.001 | 1.7% |
| 4 | 0.046 ± 0.001 | 1.6% | 0.045 ± 0.001 | 2.5% |
| 5 | 1.142 ± 0.016 | 1.9% | 1.147 ± 0.038 | 4.7% |
| 6 | 0.88 ± 0.009 | 1.8% | 0.861 ± 0.023 | 6.3% |
| 7 | 0.341 ± 0.006 | 2.4% | 0.349 ± 0.003 | 1.2% |
| 8 | 0.394 ± 0.008 | 4.4% | 0.398 ± 0.00 | 1.3% |

### Example 2

Correlation between neutralizing antibodies and ELISA antibodies.

In the serum neutralization experiment, the IBV-positive serum to be determined was inactivated at 56° C. for 30 minutes, diluted in multiples, and mixed with an equal volume of 100 median tissue culture infective dose (TCID₅₀) IBV for 1 hour. A resulting mixture was added to a 96-well plate containing 80% cultured monolayer chicken embryo kidney cells for incubation for 2 h at 37° C. with 5% CO₂. A resulting supernatant was removed, 200 mL of DMEM/F12 medium containing 2% Cefsulodin (CFS) was added, and cytopathic effect (CPE) were observed 48 hours later, and the 50% neutralization titer was calculated.

The cells were fixed and detected by indirect immunofluorescence method. The ELISA titers and neutralization titers of different types of IBV were compared, and the correlation between pELISA and neutralization test was evaluated.

Serum samples immunized with different strains such as 4/91, M41, H52, QXI87 and CK/CH/2014/FJ14 virulent strain were tested.

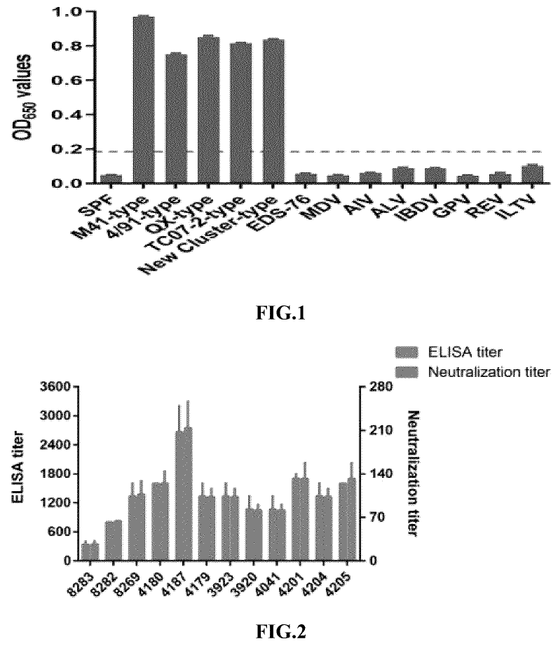

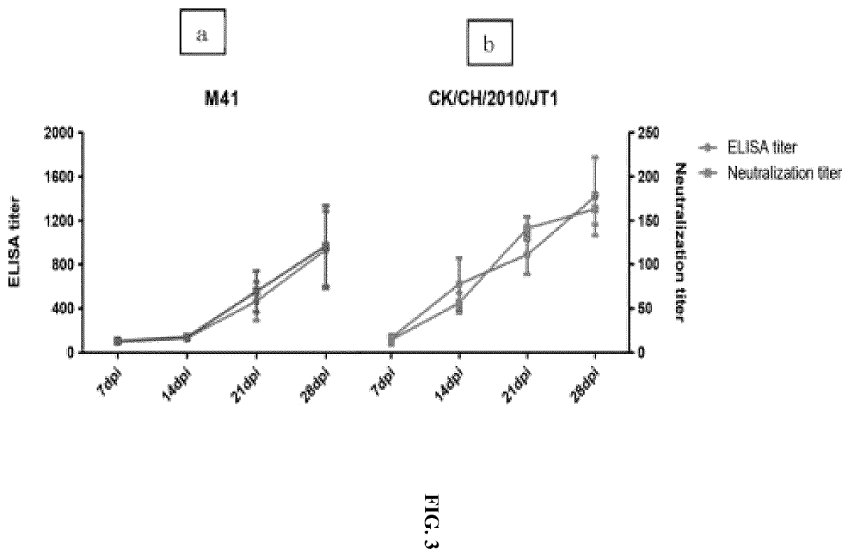

As shown in FIG. 2, the pELISA titer of serum samples 8269 (QXI87), 4179 (M41), 3923 (CK/CH/2014/FJ14) and 4204 (H52) were all 1:1333, and the neutralization titers thereof were all 1:102.

The neutralization titers of M41 sera 4179, 4180, and 4187 were 1:1333, 1:1600, and 1:2666, respectively, and the ELISA titers thereof were 1:102, 1:124, and 1:213, respectively. The results showed that the ELISA titer of the test serum was positively correlated with the neutralization titer, and it could be seen from FIG. 2 that ELISA was positively correlated with the neutralizing antibody titer.

### Example 3

Correlation of antibodies in SPF chickens infected with classic IBV strains and isolated strains at different times

In order to further evaluate the changes of infectious bronchitis virus infection and serum enzyme-linked immunosorbent assay (ELISA) and neutralizing antibody levels after vaccination, 14-day-old SPF chickens were infected with IBV CK/CH/2014/FJ14 strains and CK./CH/2010/JT1 strain or M41 strain. The serum samples of infected chicks were collected at different times, and the serum titer was determined by the pELISA method in Example 1. The result was then compared with the neutralization titer determined by the neutralization method in Example 2. As shown in FIG. 3, the ELISA titers of serum samples on the 7, 14, 21, and 28 days after the chicken inoculated with CK/CH/2010/JT1 were 1:100, 1:466.6, 1:666.6, and 1:1066, respectively. The neutralization titers of these samples were 1:14.8, 1:56.25, 1:154.5, and 1:206 (b in FIG. 3). With the increase of the infection time, the serum ELISA titer and neutralization titer both increased. In the serum collected at different time points after inoculation with M41 or CK/CH/2010/JT1, the ELISA titer and neutralization titer showed good consistency (a and b in FIG. 3). It could be seen from FIG. 3 that the neutralizing antibodies and ELISA antibodies had a good correlation after chickens were infected by M41 and CK/CH/2010/JT1.

The description of the above examples is only used to help understand the method and core idea of the present disclosure. It should be pointed out that for those of ordinary skill in the art, without departing from the principle of the present disclosure, several improvements and modifications can be made to the present disclosure, and these improvements and modifications also fall within the protection scope of the claims of the present disclosure. Various modifications to these examples are obvious to those skilled in the art, and the general principles defined herein can be implemented in other embodiments without departing from the spirit or scope of the present disclosure. Therefore, the present disclosure will not be limited to the embodiments shown herein, but should conform to the widest scope consistent with the principles and novel features disclosed herein.

1. An antigen peptide with an infectious bronchitis virus (IBV)-specific neutralizing epitope, wherein the peptide is a cyclic peptide, and the amino acid sequence of the peptide is set forth in SEQ ID NO: 1 or SEQ ID NO: 2.

2. An IBV-specific antibody, wherein the IBV-specific antibody specifically binds to the antigen peptide with the IBV-specific neutralizing epitope according to claim 1.

3-6. (canceled)

7. An ELISA method as an alternative to neutralization potency assay, comprising the following steps:
1) coating the antigen peptide with the IBV-specific neutralizing epitope on an ELISA plate, and blocking it with rabbit serum after coating;
2) after washing for several times, adding chicken serum for co-incubation;
3) washing again for several times, then incubating with an enzyme-labeled goat anti-chicken IgG antibody, and after washing for several times, using tetramethylbenzidine (TMB) substrate to develop color; and
4) terminating the reaction with 0.1% sodium dodecyl sulfate (SDS), and detecting an absorbance at 650 nm with an enzyme-linked immunosorbent analyzer to determine a neutralizing antibody level against IBV in an individual.

8-12. (canceled)
